(19) United States
(12) Patent Application Publication
(10) Pub. No.: US 2003/0225031 A1
(43) Pub. Date: Dec. 4, 2003

(54) Administration of acetylcholinesterase inhibitors to the cerebral spinal fluid

(76) Inventor: Steven C. Quay, Edmonds, WA (US)

Correspondence Address: Nastech Pharmaceutical Company Inc., 3450 Monte Villa Parkway, Bothell, WA, 98021-8906, US

(21) Appl. No.: 10/439,108

(22) Filed: May 15, 2003

Related U.S. Application Data
(60) Provisional application No. 60/382,122, filed on May 21, 2002.

(51) Int. Cl. A61K 31/724; A61K 31/55; A61K 31/48; A61K 31/473

(52) U.S. Cl. 514/55; 514/297; 514/478; 514/214.01; 514/58; 514/458; 514/379; 514/217.04; 514/288; 424/45

(57) ABSTRACT

Methods and compositions are disclosed that provide acetylcholinesterase inhibitors for the prevention and treatment of diseases and disorders of the central nervous system, including dementia such as Alzheimer's disease, to the central nervous system via intranasal delivery. The methods and compositions of the present invention provide therapeutic concentrations of the acetylcholinesterase inhibitor in the cerebrospinal fluid of a mammal without the attendant disadvantages, risks and side effects of oral or injection delivery.

This claims priority under 35 U.S.C. §119 (e) to U.S. Provisional Application No. 60/382,122 filed May 21, 2002, the entire contents of which are incorporated herein by reference.

### BACKGROUND OF THE INVENTION

The teachings of all of the references cited herein are incorporated herein by reference.

Acetylcholinesterase inhibitors are an important class of drugs for the prevention and treatment of diseases and disorders of the central nervous system. These diseases include, inter alia, neurological conditions associated with memory loss, cognitive impairment and dementia in mammals, including Alzheimer's Disease, Parkinson's-type dementia, Huntington's-type dementia, Pick's-type dementia, CJ-type dementia, AIDS-related dementia, Lewy Body dementia, Rett's syndrome, epilepsy, brain malignancies or tumors, cognitive disorder associated with multiple sclerosis, Down's syndrome, progressive supranuclear palsy, certain forms of schizophrenia, depression, mania and related psychiatric conditions, Tourette's syndrome, mysasthenia gravis, attention deficit disorder, autism, dyslexia, forms of delirium, or dementia as a sequela to vascular stroke or cranial bleeding and brain injury, in their chronic, acute and relapsing forms. Pathological changes in Alzheimer's disease, for example, involve degeneration of cholinergic neurons in the subcortical regions and of neuronal pathways that project from the basal forebrain, particularly Meynert's nucleus basalis to the cerebral cortex and hippocampus (Robert P H et al. 1999. “Cholinergic Hypothesis and Alzheimer's Disease: The Place of Donepezil (Aricept),” Encephale 5:23-5 and 28-9). These pathways are thought to be intricately involved in memory, attention, learning, and other cognitive processes.

The earliest signs of dementia appear as mild cognitive and memory impairment. This occurs progressively in underlying conditions such as Alzheimer's disease and suddenly in dementia related to vascular embolism or bleeding aneurism. Dementia in its advanced form is associated with aggressive behavior, irrational and paranoid ideation, loss of memory, loss of sense of smell, and often with cataracts. Non-vascular dementia is always related to abnormal deposition of a particular protein in the nerve bodies and sheaths. In Alzheimer's the abnormal deposits of amyloid protein are called plaques. Plaques containing other unique proteins appear in Parkinson's disease, Huntington's disease, Pick's disease and in prion disease associated with cognitive impairment. These plaques can be identified histologically at autopsy. Dense tangles of the tau protein are also observed intracellularly in dementia. Certain alleles at several loci, most notably the ApoE e4 allele, have been noted to have a higher incidence of late-onset dementia. Late and early onset forms of the disease are differentiated by the age at onset; 65 years of age can be taken as a cutoff for “early” onset versus late onset disease.

Vascular dementia may present with unique symptoms, such as gait abnormality and urinary incontinence, generally due to multiple cerebral infarctions, intracerebral hemorrhage, strokes, or infectious vasculitis of Lyme's disease, and autoimmune vasculitis of lupus erythrematosis, among other related conditions.

Of all testing methods for dementia and delerium, the most objective early measure is cognitive testing. Standardized testing in humans may be performed using the Reye Auditory Verbal Learning Test, the Mini-Mental State Exam (MMSE) the Weschler Logical Memory Test, or the Selective Reminding Test, among others. The cognitive subscale is also a major indication in the Alzheimer's Disease Assessment Scale (ADAS-cog), and simultaneously assesses short term memory, orientation in place and time, attention span, verbal ability and praxis. ADAS-cog testing is used diagnostically, higher scores indicating cognitive impairment, but may also be used to evaluate success in treatment. Reduced scores following treatment with tacrine, donepezil and the longer-acting rivastigmine have been noted.

It is believed that acetylcholinesterase inhibitors exert their therapeutic effect in the central nervous system by enhancing cholinergic function, i.e., by increasing the concentration of acetylcholine through reversible inhibition of its enzymatic hydrolysis by the cholinesterases. This pharmacotherapeutic approach also has some value in treatment of nicotine withdrawal and sleep apnea, as well as the dementia and delerium states described above. The three acetylcholinesterase inhibitor drugs presently on the market are delivered orally in the form of tablets and capsules. During oral delivery, drug passes down the digestive tract and is absorbed into blood capillaries of the duodenum and ileum, enters the portal vein, and is then transported to the liver before reaching the target organ, the brain. Unfortunately, oral delivery of acetylcholinesterase inhibitors is associated with several disadvantages, including, inter alia:

(1) hepatic first pass metabolism and clearance,

(2) gastrointestinal destruction of the drug by digestive enzymes and by the acidic pH conditions of the digestive tract;

(3) inferior and unpredictable uptake and bioavailability, especially as affected by food ingestion; and

(4) serious adverse effects, including nausea, vomiting, loose stools, diarrhea, anorexia and in severe cases, irreparable esophageal tears.

A recent report of an esophageal tear in an Alzheimer's patient receiving the ACE inhibitor, rivastigmine, (Kumar V. 2001. Spontaneous rupture of oesophagus (Boerhaave's syndrome) related to rivastigmine. Age Ageing 30:177) emphasizes the danger of oral administration of these drugs in debilitated patients. Longitudinal rupture of the esophagus is often not surgically repairable.

The possibility of injection or topical application of a cholinesterase has been superficially disclosed, and sufficient information has been disclosed to enable a suitable injectable dose form of donepezil in PCT/US01/07027. However, injectables are not suitable for many patients with low muscle mass, are inherently dangerous in patients who lack a fully functional immune system, and require extra expense, time and training. When one considers that a dose of these drugs may be required up to 4 times a day, the highly invasive route of administration by injection seems a flatly unacceptable alternative unless the patient is hospitalized and has a central IV line open at all times.

Another option that has been contemplated for the delivery of acetylcholinesterase inhibitors is by inhalation and absorption via the pulmonary mucosa. This method is alluded to in PCT/US01/07027 (“Novel Methods Using Cholinesterase Inhibitors”). The disclosure discloses that aerosol sprays and fine powdered solid dosage forms can reach the lung when administered by pressurized spray or ventilatory support through the nose or mouth. These inhalation dosage forms are formulated with propellants for use in insufflators or nebulizers. However, in order to reach the large surface area of the alveoli, special equipment is often needed. The formulations require propellants or other means of achieving a very fine mist or powder with particle size less than 10 um diameter. The mist or powder is then administered to the lungs via the mouth or the nose (using intubation). If possible, the patient must be trained to actively inhale during dosing, or pressurized respiratory assist may be required, as in patients suffering from asthma, chronic obstructive pulmonary disease, emphysema or physical debilitation due to ageing. Generally, in these patients, the assistance of a trained technician is required to achieve efficacious dosing. Unfortunately, consistency of dosing with mists or powders has been problematic, and most research has been directed to high-density powders because liquid solutions of drug, especially aqueous solutions, do not have the low surface tension needed to form a dense and slow settling microaerosol suitable for inhalation therapy. The methods employed do not readily lend themselves to home dosing of elderly patients and are inherently expensive due to the costs associated with the specialized delivery devices and route of administration. Metered dose inhalation is one of the most complex drug delivery systems on the market. More information about pressurized devices used for aerosol inhalation drug delivery is provided in Remington: The Science and Practice of Pharmacy, 19^{th} ed. Chapter 95 “Aerosols”, and a descriptive definition of the inhalation route for drug delivery is provided in Chapter 41, “Drug Absorption, Action and Disposition” under the heading of Absorption of Drugs: Inhalation Route, and is quoted herein as a definition for the nasal and oral methods of inhalation drug delivery (vide infra).

Therefore, the problem remains for delivery of acetylcholinesterase inhibitors. What is needed is a simple, improved method of delivery of acetylcholinesterase inhibitors for the prevention and treatment of diseases and disorders, e.g., of the central nervous system that avoids the toxicity and low bioavailability associated with oral delivery, and the expense, training and difficulty of dosing by injection and inhalation therapy. The methods and compositions of the present invention meet this urgent need.

There have been attempts to deliver other drugs intranasally, i.e. without formulation or devices for inhalation, in the treatment of brain disorders. For example, U.S. Pat. No. 6,180,603 discloses a method for actively and transaxonally delivering therapeutic agents which are the autologous counterparts of endogenous proteins, peptides and complex lipids, all of which are native to the brain. Delivery of this class of drugs is accomplished via interneuronal transport in nerve cell bodies and membranes. Specifically, the patent discloses the transport of insulin, insulin-like growth factors, nerve growth factors, gangliosides, phosphatidylserine, brain-derived neurotrophic factors, fibroblast growth factors, glia-derived nexins, ciliary neurotrophic factors and cholinergic enhancing factors via the axon of the olfactory nerve. However, there is no disclosure or teaching of any useful intranasal delivery by this mechanism of non-naturally occurring, xenogenic drugs including synthetic heterocyclic amines, substituted piperidines, and substituted phenols, further encompassing acetylcholinesterase inhibitors, and, in particular, xenogenic, non-native acetylcholinesterase inhibitors. No disclosure is made of excipients useful for increasing paracellular and transcellular uptake.

It was wholly unexpected that the pharmaceutical compositions of the invention containing acetylcholinesterase inhibitors and at least one permeation-enhancement agent could be delivered in efficacious quantity via the intranasal administration methods disclosed herein because these cationic drugs are not native to the body, and there would have been no expectation of preferential delivery to the brain or any organ, except to the liver for metabolism and excretion.

The uptake I describe for ACE inhibitors is substantially paracellular. And because the enhancement is paracellular, the drug rapidly enters the CSF and blood where it is distributed throughout the brain. Data collected with apomorphine as a small molecule xenogenic model drug shows elevated concentrations in cortex, midbrain, medulla oblongata, cerebellum and CSF. CSF data for apomorphine is higher than expected from comparative studies with oral or intravenous administration, possibly by the subarchnoid plexus, although this is speculative.

The present invention is directed to a method and composition that provides the acetylcholinesterase inhibitors to the central nervous system via paracellular intranasal delivery. The rich vascular plexus of the nasal cavity of a mammal provides a direct route into the bloodstream for the acetylcholinesterase inhibitors that readily cross mucous membranes. Due to the direct absorption into the bloodstream, problems of gastrointestinal destruction and hepatic first pass metabolism are avoided, thereby improving the bioavailability of the drug relative to oral delivery. The method and composition of the present invention provide a higher bioavailability and maximum concentration in the central nervous system of the mammal relative to other simple modes of delivery, without the attendant disadvantages and side effects of oral or injectable dosing. Specifically, the serious gastrointestinal problems associated with present methods of oral delivery are avoided.

The pharmaceutical compositions of the present invention minimize the transport of the acetylcholinesterase inhibitors from the nasal passages into the lungs. As is well known in the art, acetylcholinesterase inhibitors have some crossreactivity with butylcholinesterase inhibitors. Butylcholinesterases are a structurally-related enzyme family, but have very different biological functions. Whereas inhibition of acetylcholinesterase can lead to beneficial accumulation of acetylcholine in synapses, inhibition of butylcholinesterase can result in respiratory failure, especially when the inhibitor enters the lung. This toxicity forms the basis for the widely known use of butylcholinesterase inhibitors as poisons and insecticides. Therefore, the methods of the present invention, wherein nasal application is proposed for commercial use in treatment of disease, are specifically designed to restrict contact of the formulation to the nasal turbinates and oropharynx. Droplet size of sprays are larger than about 20 to 100 um, so that the spray droplets immediately drop to the nasal mucosa and do not enter the lungs as an aerosol. While a few droplets potentially can escape and enter the oropharynx, essentially no material will enter the lungs in the form of an aerosol. Gel formulations for intranasal application are also applied with simple pump or squeeze devices and do not permit acetylcholinesterase inhibitor to enter the lungs. Dose volume is typically limited to less than 0.9 mL per nostril, more preferentially to 0.2 mL per nostril, and most preferentially to less than or equal to 0.1 mL per nostril. Nose droplets may also be used without risk. Therefore, the products are designed to be safe and do not lead to toxic poisoning, as is an evident possibility with nasal or oral inhalation methods when used with acetylcholinesterase inhibitors.

### BRIEF DESCRIPTION OF THE DRAWINGS

FIG. 1 shows a plot of Donepezil cereberal spinal fluid/plasma ratio in a rat, following single-dose nasal administration at time zero, followed by dual measurements of CSF and plasma at 5, 10, 15, 30 and 60 minutes.

### BRIEF SUMMARY OF THE INVENTION

In a first embodiment, the invention is directed to a pharmaceutical composition for the for treatment or prevention of a disease or condition in a mammal in need of treatment by therapeutic administration of an acetylcholinesterase inhibitor, comprising:

a. a liquid or gel solution for nasal administration of at least one acetylcholinesterase inhibitor; and

b. at least one, preferably a plurality of, permeation-enhancement agents for transmucosal drug uptake.

These diseases include Alzheimer's disease and other neurological conditions associated with cognitive impairment in a mammal.

Preferably, the acetylcholinesterase inhibitor is donepezil, tacrine, rivastigmine or a pharmaceutically-acceptable salt or derivatives thereof. Preferably, the acetylcholinesterase inhibitor is substantially free of native neurobiomolecules. In some embodiments, the acetylcholinesterase inhibitor is administered to the mammal in an effective dose of between about 0.1 mg to about 100 mg per dose, and up to 6 doses per day, more preferentially about 1 to 50 mg per dose, and most preferentially 1.5 to 12 mg per dose. Dosage is given preferably once per day, but acceptably four times per day or more. Dosing may have to be gradually increased to develop tolerance. The dosage regime is expected to be dependent on the degree and severity of symptomatology, body weight, the presence or absence of renal failure or cirrhosis, and other factors that may be evaluated by the attending physician or veterinarian, and may vary widely.

In another preferred embodiment, the pharmaceutical composition of the invention following intranasal adminstration to a mammal yields a peak concentration of the acetylcholinesterase inhibitor in a central nervous system tissue or fluid of the mammal that is at least equal to, preferably at least 10%, 15%, 20%, 25%, 30%, more preferably 35% and most preferably 40% greater than the therapeutic plasma concentration of the acetylcholinesterase inhibitor in a blood plasma of the mammal. Improvements on this are expected with further research.

In another embodiment, the invention is directed to a method for treating or preventing a disease or condition in a mammal in need of treatment by therapeutic administration of an acetylcholinesterase inhibitor, including the step of administering a pharmaceutical composition containing:

a. a liquid or gel solution for nasal administration of at least one acetylcholinesterase inhibitor; and

b. at least one, preferably a plurality of, permeation-enhancement agents for transmucosal drug uptake.

Preferably, the components of the pharmaceutical composition are administered simultaneously, consecutively, or separately. Preferably, the pharmaceutical composition is administered as a single solution. Most preferentially, the components of the composition are administered intranasally.

In other embodiments, the invention is directed to an administering device, preferably a nasal dispenser or pump for use with said composition, which may optionally be a multidose device. In further embodiments, the invention is directed to an article of manufacture comprising the pharmaceutical composition of the invention in a childproof package suitable for sale and distribution.

As used herein, the following definitions are provided as an aid in interpreting the claims and specification herein. Where works are cited by reference, and definitions contained therein are inconsistent with those supplied here, the definition used therein shall apply only to the work cited and shall not be applied to this disclosure.

“Mammal” shall include any of a class of warm-blooded higher vertebrates that nourish their young with milk secreted by mammary glands and have skin usually more or less covered with hair, and non-exclusively includes humans and non-human primates, their children, including neonates and adolescents, both male and female, livestock species, such as horses, cattle, sheep, and goats, and research and domestic species, including dogs, cats, mice, rats, guinea pigs, and rabbits. “Patient” or “subject” is used herein interchangeably with “mammal.”

“Dementia” shall mean a broad deterioration of intellectual functioning with impaired or absence of clarity in conscious awareness, and is characterized by one or more symptoms of impaired short term memory, impaired judgment, impaired rational intellect, and/or disorientation with respect to place or time. Dementia is considered “irreversible” when, as is typical, accompanied by organic brain disease. Dementia is always associated with disability in the conduct of an independent lifestyle. The symptoms of dementia encompass but are worse than those of cognitive impairment.

“Cognitive impairment” is a disorder in memory, problem solving, abstract reasoning and orientation that weakens an individual's ability to maintain an independent lifestyle. Mild cognitive impainnent does not rise to the level of Alzheimer's disease and is not unusual in ageing. A hallmark is memory impairment with resulting confusion in the conduct of daily affairs.

“Intranasal delivery” shall mean delivery of a drug primarily via the mucosa of the nasal cavity. This includes the superior, middle and inferior nasal turbinates and the nasal pharynx. Note that the olfactory region is concentrated in the superior (upper ⅓) of the nasal turbinates. Cilial action pushes material back toward the oropharynx, so material deposited in the nasal vestibule encounters the nasal mucosa before entering the throat.

“Acetylcholinesterase inhibitor” shall mean a xenogenic or naturally-occurring compound that increases the concentration of acetylcholine through reversible inhibition of its hydrolysis by acetylcholinesterase.

“Native neurobiomolecule” refers to any cellular or humoral signal molecule that is genetically encoded for by a mammal or is formed in the normal metabolism of a mammal, and is found in normal mammals in the brain or CSF. By way of example, native neurobiomolecules include ganglioside, phosphatidylserine, brain-derived neurotropic factor, fibroblast growth factor, insulin, insulin-like growth factors, ciliary neurotropic factor, glia-derived nexin, cholinergic enhancing factors, phosphoethanolamine, and thyroid hormone T3. It is thought that lipids such as phosphatidylserine are components of a vesicular transport mechanism specific for these molecules. In contrast, note that the acetylcholinesterase inhibitors that are the subject of this invention are ex vivo synthetic, non-naturally occurring chemicals herein termed “xenogenic molecules” or “xenogenic acetylcholinesterase inhibitors”.

“Xenogenic” refers herein to any synthetic product of man's chemical art and skills which is not found in nature as the natural product of a biosynthetic pathway of a mammal.

“Naturally occurring” refers to biomolecules that are extracted or derivatized from plant or animal sources, including oleagenous and petroleum deposits.

“Inhalation route”: As described in Remington's, “Inhalation may be employed for delivering gaseous or volatile substances into the systemic circulation, as with most general anesthetics. Absorption is virtually as rapid as the drug can be delivered into the alveoli of the lungs, since the alveolar and vascular epithelial membranes are quite permeable, blood flow is abundant and there is a very large surface area for absorption. Aerosols of nonvolatile substances also may be administered by inhalation, but the route is used infrequently for delivery into the systemic circulation because of various factors that contribute to erratic and difficult-to-achieve blood levels. Whether or not an aerosol reaches and is retained in the pulmonary alveoli depends critically upon particle size. Particles greater than 1 um in diameter tend to settle in the bronchioles and bronchi, whereas particles less than 0.5 um fail to settle and are mainly exhaled.” MR Franklin. “Drug Absorption, Action and Disposition” in Remington: The Science and Practice of Pharmacy. 19^{th} ed. pp 711-12.

“Nasal route:” Drugs may be given intranasally by direct application to the nasal mucosa lining the nasal turbinates. The mucosa is richly vascularized and enervated and extends from the nasal nares to the upper boundary of the oropharynx and the lower boundaries of the sinus passages. Drugs applied to the nasal mucosa permeate trans-mucosally by either paracellular diffusion (passive), or by transcellular diffusion (passive or facilitated diffusion, or active transport). Passive diffusion is most conveniently employed for molecules less than 1 kilodaltons in size, perhaps up to 5 kilodaltons as a maximum for any substantial uptake. However, uptake by the nasal route of administration is not so limited. Nasal uptake of peptides and proteins of up to several hundred kilodaltons has been demonstrated in the past few years. Drug crossing the mucosal barrier enters nasal capillaries and the general circulation, bypassing the liver on the first pass. It is thought that drug may also enter the olfactory or trigeminal nerve bundle and be transported intra-axonally to the central nervous system (CNS). It is further speculated that drug may also diffuse into the cerebrospinal fluid (CSF) by the subarachnoid plexus and is transported in the flow of the CSF to other parts of the brain and spinal cord. Thus the nasal route of administration has multiple pathways and special relevance to drugs that target the CNS.

“Pharmaceutically acceptable” refers to a composition which, when administered to a human or a mammal by the indicated route of administration, provokes no adverse reaction which is unacceptably disproportionate to the benefit gained by administration of said compound. Note that the therapeutic substance may be the source of the adverse reaction, but in some cases substitution of other excipients may modulate this toxicity. And when the excipient itself is the source of the toxicity, reformulation will remove the discomfort. Excipients required for one route of drug delivery may not be required for an alternate route. Thus the art of drug formulation encompasses both choice of route of drug delivery and choice of excipient.

“Delivery system” refers to a combination of excipients used to promote and make pharmaceutically acceptable a formulation (with or without a device) used to deliver a measured dose or sufficient quantity of a drug by the chosen route of administration.

“Permeation-enhancement agent” refers to excipients in a composition for intranasal drug administration that enhance overall delivery, dose consistency and/or pharmaceutical acceptance. The methods for assessing enhancement are quantitative and are defined in the examples herein. Functional in vitro cell-based testing and methods are used to compare the effects of drug molecule by itself versus the same mass of drug molecule formulated and administered with excipients. “Enhancement” is a multivariant value proposition encompassing total bioavailability, net uptake, consistency of uptake and dosing, drug metabolism, degradation during dosing, drug targeting to active site(s), mucosal irritation, and overall safety and toxicity. In some cases, the relative benefit of the drug is used to optimize the excipient composition of the formulation, even at the expense of factors such as comfort and side effects. As explained when “pharmaceutical acceptance” was defined, the benefits of therapy may outweigh the discomfort of delivery. Chronic administration must also be considered. However, comfort and safety are optimized when overall delivery is not unacceptably compromised. As an illustration of the balance, pH may improve the stability of a drug and its transmucosal flux, but a formulation pH outside the range of pH 3 to 8.5 may result in pain and tissue damage when administered nasally. Thus there is always a balance to be struck in formulating excipients.

“About” is a relative term denoting an approximation of plus or minus 20% of the nominal value it refers to. For the field of pharmacology and clinical medicine and analogous arts that are the subject of this disclosure, this level of approximation is appropriate unless the value is specifically stated to be critical or to require a tighter range.

“Substantially free” refers to the level of a particular active ingredient in the compositions of the invention, wherein the particular active ingredient constitutes less than 20%, preferably less than 10%, more preferably less than 5%, and most preferably less than 1%, by weight based on the total weight of active ingredients in the composition.

“Degradant” refers to a product of a chemical reaction occurring in vitro during storage, dissolution, or upon stability testing under stress from heat, light, co-solutes and solvent, generally refers to a drug substance, and is contrasted with a metabolite.

“Metabolite” refers a product of a chemical or enzymatic reaction occurring in vivo, generally refers to a drug substance, and is contrasted with a degradant.

“Nasal mucosa” refers to the lining of the nasal cavity, where vascularized, and extending interiorly to the boundaries of the oropharynx and sinuses. It includes the superior turbinate, the middle turbinate, and the inferior turbinate. It is the latter two where most of the non-olfactory epithelium is found in man.

“Liquid” refers to a solution or formulation in the liquid state. Note that not all liquids are aqueous and that liquid behavior is not infrequently temperature dependent.

“Aqueous” refers to a solution formed in water, but may contain lesser amounts of other co-solvents. Note that not all aqueous solutions are liquids.

“Gel” refers to a thickened solution, aqueous or otherwise, used as a drug delivery vehicle and may be administered as a spray or an ointment when given intranasally.

“Short chain” refers to carbon lengths of C₁₂ or less, where the chain may be aliphatic or branched.

“Stability” or “stability during storage” refers to any compositional change measured in a parameter as a function of a commercially relevant time interval and conditions of storage, for example concentration, degradation, viscosity, pH, or particle size, which is greater than 10% over a time interval relevant to commercial shelf life, denotes instability. Changes less than or equal to 10% connote stability. The time period over which stability is measured is relative depending on the intended commercial distribution and storage conditions for the composition. “Accelerated stability testing” at higher temperature is sometimes taken as a more speedy way of extrapolating stability over longer periods of time. For example, a 4-month study at 40° C. can be taken to predict stability at controlled room temperature for over one year.

Three modes of drug uptake and transport are distinguished in the text:

“Paracellular” is used in its classical sense to indicate transport of a molecule between the cells of an epithelium, as in the nasal mucosa. Mannitol is taken as the reference permeant, and transport is passive, dependent on the size of the molecule and the size of the water channels in the cell junctions. This diameter is dependent on treatment of the membrane with enhancers that weaken the tight junctions or expand water channels.

“Transcellular” is used to indicate other forms of uptake at an epithelium that may be passive, facilitated diffusion, or active. This category includes diffusion of lipidic drugs in the membrane of epithelial cells from apical to basolateral sides, followed by escape of the drug by exchange or by blebbing into the cytosol. This category may include non-specific endocytosis and vesicular transport.

“Transaxonal” refers to specific uptake of native signaling molecules, defined here as neurobiomolecules, for tubulin-mediated active transport within nerve axons. This transport is typically vesicular and is believed to rely on receptor-mediated endocytosis. The phenomenon, first documented by Maitani in the rabbit nasal mucosa, is specialized for cytokines, hormones and the special lipid components that make up the vesicles formed for transport via this pathway.

The methods of the invention provide at least one acetylcholinesterase inhibitor to the central nervous system of a mammal via intranasal delivery, by applying to the nasal cavity of the mammal a pharmaceutical composition comprising:

a. a liquid or gel solution, preferably an aqueous solution, of at least one acetylcholinesterase inhibitor; and

b. at least one permeation-enhancement agent. The method of the invention improves the bioavailability of the acetylcholinesterase inhibitor to the central nervous system because the delivery route bypasses the digestive tract, liver and lungs.

The pharmaceutical compositions of the invention useful for the prevention and/or treatment of diseases and disorders of the central nervous system, include:

a. a liquid or gel solution, preferably an aqueous solution, of at least one acetylcholinesterase inhibitor; and

b. at least one permeation-enhancement agent;

wherein the pharmaceutical composition is suitable for intranasal delivery.

The methods and compositions of the invention are suitable for the prevention and treatment of diseases and disorders of the central nervous system, including, for example, neurological conditions associated with memory loss and cognitive impairment in mammals, including Parkinson's-type dementia, Huntington's-type dementia, Pick's-type dementia, CJ-type dementia, AIDS-related dementia, Lewy Body dementia, Rett's syndrome, epilepsy, brain malignancies or tumors, cognitive disorder associated with multiple sclerosis, Down's syndrome, progressive supranuclear palsy, certain forms of schizophrenia, depression, mania and related psychiatric conditions, Tourette's syndrome, mysasthenia gravis, attention deficit disorder, autism, dyslexia, forms of delirium, or dementia as a sequela to vascular stroke or cranial bleeding and brain injury, in their chronic, acute and relapsing forms. Nicotine withdrawal is also a condition that is treated with acetylcholinesterase inhibitors.

Suitable acetylcholinesterase inhibitors include xenogenic compounds such as donepezil, 6-O-desmethyl donezepil, tacrine, rivastigmine, neostigmine, pyrrdostigmine, physostigmine, ipidacrine, stacofylline, galantamine, galanthamine analogs, lycoramine, lycoramine analogs, physostigmine, ambenonium, demecarium, eprophonium, metrifonate, selegine, metrifonate, 3-[1-(phenylmethyl) piperidine-4-yl]-1-(2,3,4,5-tetrahydro-1H-1-benzazepine-8-yl)-1-propane, 5,7-dihydro-3-[2-(1-(phenylmethyl)-4-pyperidinyl)ethyl]-6H-pyrrolo-[4,5-f]-1,2-benzisoxazole-6-one, 4,4′-diaminodiphenylsulfone, tetrahydroisoquinolinyl carbamate of pyrroloindole derivative and pharmaceutically-acceptable salt or derivative thereof.

Pharmaceutically-acceptable salts include inorganic acid salts, organic amine salts, organic acid salts, alkaline earth metal salts and mixtures thereof. Suitable examples of pharmaceutically-acceptable salts include, but are not limited to, halide, glucosamine, alkyl glucosamine, sulfate, hydrochloride, carbonate, hydrobromide, N,N′-dibenzylethylene-diamine, triethanolamine, diethanolamine, trimethylamine, triethylamine, pyridine, picoline, dicyclohexylamine, phosphate, sulfate, sulfonate, benzoate, acetate, salicylate, lactate, tartate, citrate, mesylate, gluconate, tosylate, maleate, fumarate, stearate and mixtures thereof.

In certain preferred embodiments, the compositions and methods may further include a COX-2 inhibitor, huperzine (selegine) or 4,4′-diaminodiphenylsulfone

In combination with the acetylcholinesterase inhibitors, the pharmaceutical compositions useful in the invention include at least one excipient. Authoritative reviews of the pharmaceutical arts with respect to intranasal formulations are provided by Behl and Chien (Behl, CR et al. 1998. “Effects of physicochemical properties and other factors on systemic nasal drug delivery,” Adv Drug Del Rev 29:89-116; Behl CR et al. 1998. “Optimization of systemic nasal drug delivery with pharmaceutical excipients,” Adv Drug Del Rev 29:117-133 and, Chien, YW. 1992. 2^{nd} Ed. Novel Drug Delivery Systems, Marcel Dekker, NY).

The pharmaceutical compositions useful in the invention include at least one permeation-enhancement agent. As used herein and as defined in more detail elsewhere, “permeation-enhancement agent” includes agents which enhance the release or solubility of the drug (e.g., from a formulation delivery vehicle), diffusion rate, bioavailability, penetration capacity and timing, uptake, residence time, stability, effective half-life, peak or sustained concentration levels, clearance, reduction of irritation, comfort, biotolerance, and other desired intranasal cavity delivery characteristics (e.g., as measured at the site of delivery, or at a selected target site of activity in the central nervous system) of the acetylcholinesterase inhibitor. Enhancement of intranasal cavity delivery can thus occur by any of a variety of mechanisms, for example by increasing the diffusion, transport, persistence or stability of acetylcholinesterase inhibitor, increasing membrane fluidity in the epithelium, increased fluidity of the mucous secretions, modulating the availability or action of calcium and other ions that regulate intracellular or paracellular permeation, solubilizing mucosal membrane components (e.g., lipids), changing non-protein and protein sulfhydryl levels in nasal mucosal layer, increasing water flux across the nasal mucosal surface, modulating epithelial junctional physiology, reducing the viscosity of mucus overlying the nasal mucosal epithelium, reducing nasal mucociliary clearance rates, and other mechanisms.

Suitable permeation-enhancement agents include:

a. an aggregation inhibitory agent;

b. a charge modifying agent;

c. a pH control or pH buffering system;

d. a redox control or redox ‘buffering’ system

e. a degradative enzyme inhibitory agent;

f. a mucolytic or mucus clearing agent;

g. a ciliostatic agent;

h. an absorption enhancing agent or system selected from the group consisting of:

(i) a surfactant;

(ii) a bile salt;

(ii) a phospholipid additive, mixed micelle, liposome, or carrier system;

(iii) an alcohol;

(iv) an enamine;

(v) a nitric oxide donor compound;

(vi) a long-chain amphipathic molecule;

(vii) a small hydrophobic uptake enhancer;

(viii) sodium or a salicylic acid derivative;

(ix) a glycerol ester of acetoacetic acid;

(x) acyclodextrin or □-cyclodextrin derivative;

(xi) a medium-chain or short-chain fatty acid;

(xii) a chelating agent;

(xiii) an amino acid or salt thereof,

(xiv) an N-acetylamino acid or salt thereof;

(xv) an enzyme degradative to a selected membrane component;

(ix) an inhibitor of fatty acid synthesis;

(x) an inhibitor of cholesterol synthesis;

(i) a modulatory agent of epithelial junction physiology;

(j) a vasodilator agent;

(k) a stabilizing delivery vehicle, carrier, support or complex-forming species with which the acetylacetylcholinesterase inhibitor is effectively combined, associated, contained, encapsulated or bound resulting in complexing or stabilization of said acetylcholinesterase inhibitor for enhanced intranasal delivery; and

(l). a humectant or other anti-irritant.

Aggregation inhibitory agents include, among others, surfactants, salts such as NaCl, KCl, and sugars, particularly poloxamers that limit close approach of particles or reduce zeta potential between charged elements that would otherwise flocculate. pH adjustment is typically done using TAC grade reagent NaOH or HCl. When buffering capacity is desired, a buffering system having a pK near the desired pH is selected. Buffering systems well accepted for topical pharmaceuticals include acetate, citrate, phosphate (at 4 and 7), imidazole, histidine, glycine, tartrate and TEA. Acids for pH adjustment and salt formation include hydrochloric acid, hydrobromic acid, hydriodic acid, sulfuric acid, carbonic acid, nitric acid, boric acid, phosphoric acid, acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and mixtures thereof. Bases for pH adjustment and salt formation include basic amino acids, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, diethanolamine, triethanolamine, triethylamine, and triisopropanolamine.

Redox control and redox “buffering” agents include ascorbic acid, ascorbyl palmitate, any of the tocopherols, alpha, beta, gamma, delta, and mixed tocopherols, and the corresponding tocotrienols,

Degradative enzyme inhibitory agent include PMSF, amastatin, bestatin, trypsin inhibitor, camostat mesilate, and boroleucine. P-aminobenzamidine, FK-448, camostat mesylate, sodium glycocholate, an amino acid, a modified amino acid, a peptide, a modified peptide, a polypeptide protease inhibitor, a complexing agent, a mucoadhesive polymer, a polymer-inhibitor conjugate, or a mixture thereof, aminoboronic acid derivatives, n-acetylcysteine, bacitracin, phosphinic acid dipeptide derivatives, pepstatin, antipain, leupeptin, chymostatin, elastatin, bestatin, hosphoramindon, puromycin, cytochalasin potatocarboxy peptidase inhibitor, amastatin, protinin, Bowman-Birk inhibitor, soybean trypsin inhibitor, chicken egg white trypsin inhibitor, chicken ovoinhibitor, human pancreatic trypsin inhibitor, EDTA, EGTA, 1,10-phenanthroline, hydroxychinoline, polyacrylate derivatives, chitosan, cellulosics, chitosan-EDTA, chitosan-EDTA-antipain, polyacrylic acid-bacitracin, carboxymethyl cellulose-pepstatin, polyacrylic acid-Bowman-Birk inhibitor, and mixtures thereof are also contemplated as enzyme inhibitory substances. (See, Bernkop-Schnurch, “The use of inhibitory agents to overcome the enzymatic barrier to perorally administered therapeutic peptides and proteins,” Journal of Controlled Release, 52:1-16)

Ciliostatic agents include preservatives such as benzalkonium chloride, EDTA, and surfactants such as bile salts, betaine, and quaternary ammonium salts.

Mucolytic agents include dithiothreitol, cysteine, methionine, threonine, s-adenosyl-methionine.

Absorption enhancing agents are selected from the groups consisting of bile acids, bile salts, ionic, nonionic zwitterionic, anionic, cationic, gemini pair surfactants, phospholipids, alcohols, glycyrrhetinic acid and its derivatives, enamines, salicylic acid and sodium salicylate, acetoacetate glycerol esters, dimethylsulfoxide, n-methylpyrrolinidinone, cyclodextrins, medium chain fatty acids, short chain fatty acids, short and medium chain diglycerides, short and medium chain monoglycerides, short chain triglycerides, calcium chelators, amino acids, cationic amino acids, homopolymeric peptides, cationic peptides, n-acetylamino acids and their salts, degradative enzymes, fatty acid synthesis inhibitors, cholesterol synthesis inhibitors.

Absorption enhancers are screened on a case-by-case basis to determine the most suitable candidate. Various models have been studied, for example those of LeCluyse and Sutton (1997. “In vitro models for selection of development candidates. Permeability studies to define mechanisms of absorption enhancement” Advanced Drug Delivery Reviews, 23:163-183). In vitro methods with the EpiAirway model have proven to be valuable.

Enhancers include PEG-fatty acid esters having useful surfactant properties. Among the PEG-fatty acid monoesters, esters of lauric acid, oleic acid, and stearic acid are especially useful. Surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. Polyethylene glycol (PEG) fatty acid diesters are also suitable for use as surfactants in nasal formulations. Hydrophilic surfactants include PEG-20 dilaurate, PEG-20 dioleate, PEG-20 distearate, PEG-32 dilaurate and PEG-32 dioleate. In general, mixtures of surfactants are also useful in the present invention, including mixtures of two or more commercial surfactant products. Several PEG-fatty acid esters are marketed commercially as mixtures or mono- and diesters. Suitable PEG glycerol fatty acid esters are PEG-20 glyceryl laurate, PEG-30 glyceryl laurate, PEG-40 glyceryl laurate, PEG-20 glyceryl oleate, and PEG-30 glyceryl oleate.

The reaction of alcohols or polyalcohols with a variety of natural and/or hydrogenated oil yields a large number of surfactants of different degrees of hydrophobicity or hydrophilicity. Commonly, the oils used are castor oil or hydrogenated castor oil, or an edible vegetable oil such as corn oil, olive oil, peanut oil, palm kernel oil, apricot kernel oil, or almond oil. Alcohols include glycerol, propylene glycol, ethylene glycol, polyethylene glycol, maltol, sorbitol, and pentaerythritol. Among these alcohol-oil transesterified surfactants, hydrophilic surfactants are PEG-35 castor oil (Incrocas-35), PEG-40 hydrogenated castor oil (Cremophorg RH 40), PEG-25 trioleate (TAGAT® TO), PEG-60 corn glycerides (Crovolg M70), PEG-60 almond oil (Crovol A70), PEG-40 palm kernel oil (Crovol PK70), PEG-50 castor oil (Emalex C-50), PEG-50 hydrogenated castor oil (Emalex® HC-50), PEG-8 caprylic/capric glycerides (Labrasolg), and PEG-6 caprylic/capric glycerides (Softigen® 767). Hydrophobic surfactants in this class include PEG-5 hydrogenated castor oil, PEG-7 hydrogenated castor oil, PEG-9 hydrogenated castor oil, PEG-6 corn oil (Labrafil 2125 CS), PEG-6 almond oil (Labrafil® M 1966 CS), PEG-6 apricot kernel oil (Labrafil 1944 CS), PEG-6 olive oil (Labrafil® M 1980 CS), PEG-6 peanut oil (Labrafil 1969 CS), PEG-6 hydrogenated palm kernel oil (Labrafil 2130 BS), PEG-6 palm kernel oil (Labrafil.2130 CS), PEG-6 triolein (Labrafil 2735 CS), PEG-8 corn oil (Labrafil WL 2609 BS), PEG-20 corn glycerides (Crovol M40), and PEG-20 almond glycerides (Crovol A40). The latter two surfactants are reported to have HLB values of about 10, which is the approximate border line between hydrophilic and hydrophobic surfactants (8 to 12 HLB). Derivatives of vitamins A, D, E, K, such as tocopheryl PEG-1000 succinate (TPGS, available from Eastman), are also suitable surfactants.

Polyglycerol esters of fatty acids are also suitable surfactants for the present invention. Among the polyglyceryl fatty acid esters, hydrophobic surfactants include polyglyceryl oleate (Plurol Oleique®), polyglyceryl-2 dioleate (Nikkol DGDO), and polyglyceryl-10 trioleate. Preferred hydrophilic surfactants include polyglyceryl-10 laurate (Nikkol Decaglyn® 1-L), polyglyceryl-10 oleate (Nikkol Decaglyn 1-0), and polyglyceryl-10 mono, dioleate (Caprol® PEG 860). Polyglyceryl polyricinoleates (Polymuls) are also preferred hydrophilic and hydrophobic surfactants. Hydrophobic surfactants include propylene glycol monolaurate (Lauroglycol® FCC), propylene glycol ricinoleate (Propymuls®)), propylene glycol monooleate (Myverol® P-O6), propylene glycol dicaprylate/dicaprate (Captex® 200), and propylene glycol dioctanoate (Captex®D 800). Included are both mono- and diesters of propylene glycol. Mixtures of propylene glycol fatty acid esters and glycerol fatty acid esters are commercially available. One such mixture is composed of the oleic acid esters of propylene glycol and glycerol (Arlacel® 186). Another class of surfactants is the class of mono- and diglycerides. These surfactants are not always hydrophobic, depending on aliphatic chain length. Surfactants in this class of compounds include glyceryl monooleate (Peceol®), glyceryl ricinoleate, glyceryl laurate, glyceryl dilaurate (Capmul® GDL), glyceryl dioleate (Capmul® GDO), glyceryl mono/dioleate (Capmul® GMO-K), glyceryl caprylate/caprate (Capmulg MCM), caprylic acid mono/diglycerides (Imwitor® 988), and mono- and diacetylated monoglycerides (Myvacet® 9-45), functioning well as absoption enhancers. Sterols and derivatives of sterols have some use in the present invention. A hydrophobic surfactant in this class is PEG-24 cholesterol ether (Solulan® C-24).

A variety of PEG-sorbitan fatty acid esters are available. In general, these surfactants are hydrophilic, although several hydrophobic surfactants of this class can be used. Among the PEG-sorbitan fatty acid esters, hydrophilic surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80).

Ethers of polyethylene glycol and alkyl alcohols are also useful as surfactants. Hydrophobic ethers include PEG-3 oleyl ether (Volpo 3) and PEG-4 lauryl ether (Brij 30). Several hydrophilic PEG-alkyl phenol surfactants are available, and are suitable for use in nasal compositions for drug delivery.

The POE-POP block copolymers are a unique class of polymeric surfactants. The unique structure of the surfactants, with hydrophilic POE and hydrophobic POP moieties in well-defined ratios and positions, provides a wide variety of surfactants suitable for use in the present invention. These surfactants are available under various trade names, including Synperonic PE series (ICI); Pluronicg series (BASF), Emkalyx, Lutrol (BASF), Supronic, Monolan, Pluracare, and Plurodac®. The generic term for these familiar polymers is “poloxamer.” Hydrophilic surfactants of this class include Poloxamers 108, 188, 217, 238, 288, 338, and 407. Hydrophobic surfactants in this class include Poloxamers 124, 182, 183, 212, 331, and 335. Surfactants of this class are commonly known as poloxamers and tetronics.

Sorbitan esters of fatty acids are suitable surfactants for use in the present invention. Among these esters, preferred hydrophobic surfactants include sorbitan monolaurate (Arlacel 20), sorbitan monopalmitate (Span-40), sorbitan monooleate (Span-80), sorbitan monostearate, and sorbitan tristearate. Esters of lower alcohols (C₂ and C₄) and fatty acids (C₈ to C₁₈) are suitable surfactants for use in the present invention. Among these esters, hydrophobic surfactants include ethyl oleate (Crodamol® EO), isopropyl myristate (Crodamol IPM), and isopropyl palmitate (Crodamol IPP).

Ionic surfactants, including cationic, anionic and zwitterionic surfactants, are suitable hydrophilic surfactants for use in the present invention. Anionic surfactants include fatty acid salts and bile salts. Cationic surfactants include carnitines such as carnityl palmitate. Specifically, preferred ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. It will be appreciated by a skilled formulator, that any pharmaceutically acceptable counterion may be used. Unlike typical non-ionic surfactants, these ionic surfactants are generally available as pure compounds rather than proprietary mixtures. These compounds are readily available from a variety of suppliers such as Aldrich, Sigma, and the like.

Particular examples of surfactants that are pH dependent include free fatty acids, particularly C₆ to C₂₂ fatty acids, and the bile acids. Ionizable surfactants include fatty acids and their salts, such as caprylic acid/sodium caprylate, oleic acid/sodium oleate, capric acid/sodium caprate; ricinoleic acid/sodium ricinoleate, linoleic acid/sodium linoleate, and lauric acid/sodium laurate; trihydroxy bile acids and their salts, such as cholic acid (natural), glycocholic acid and taurocholic acid; dihydroxy bile acids and their salts, such as deoxycholic acid (natural), glycodeoxycholic acid, taurodeoxycholic acid, chenodeoxycholic acid (natural), glycochenodeoxycholic acid, taurochenodeoxycholic acid, ursodeoxycholic acid, tauroursodeoxycholic acid, and glycoursodeoxycholic acid; monohydroxy bile acids and their salts, such as lithocholic acid (natural); sulfated bile salt derivatives; sarchocholate; fusidic acid and its derivatives; phospholipids, such as phosphatidyl choline, phosphatidyl ethanolamine, phosphatidylinositol, lysolecithin, and palmitoyl lysophosphatidyl choline; carnitines, such as palmitoyl carnitine, lauroyl carnitine and myristoyl carnitine; cyclodextrins, including alpha, beta and gamma cyclodextrins and their chemically substituted derivatives such as hydroxy propyl, 2-hydroxypropyl-□-cyclodextrin and heptakis(2,6-di-O-methyl-□-cyclodextrin and sulfobutyl ether are included here.

Ionic surfactants include the ionized form of alkyl ammonium salts; bile acids and salts, analogues, and derivatives thereof; fusidic acid and derivatives thereof, fatty acid derivatives of amino acids, oligopeptides, and polypeptides; glyceride derivatives of amino acids, oligopeptides, and polypeptides; acyl lactylates; mono-,diacetylated tartaric acid esters of mono-,diglycerides; succinylated monoglycerides; citric acid esters of mono-,diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof.

Further included are the ionized form of bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono and diacetylated tartaric acid esters of mono-,diglycerides, succinylated monoglycerides; citric acid esters of mono- and diglycerides; carnitines; and mixtures thereof. Further embodiments include PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof. Useful surfactants are the ionized forms of lecithin, lysolecithin, phosphatidylcholine, phosphatidylethanolamine, phosphatidylglycerol, lysophosphatidylcholine, PEG-phosphatidylethanolamine, lactylie esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof, with the most preferred ionic surfactants being lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

Surfactants can also be formed from alcohols; for example polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; and the un-ionized (neutral) forms of ionizable surfactants. Hydrophobic surfactants can be reaction mixtures of polyols and fatty acids, glycerides, vegetable oils, hydrogenated vegetable oils, and sterols. The hydrophobic surfactant can be selected from the group consisting of fatty acids; lower alcohol fatty acid esters; polyethylene glycol glycerol fatty acid esters; polypropylene glycol fatty acid esters; polyoxyethylene glycerides, glycerol fatty acid esters; acetylated glycerol fatty acid esters; lactic acid derivatives of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, and sterols. Lower alcohol fatty acids esters; polypropylene glycol fatty acid esters; propylene glycol fatty acid esters; glycerol fatty acid esters; acetylated glycerol fatty acid esters; lactic acid derivatives of mono/diglycerides; sorbitan fatty acid esters; polyoxyethylene vegetable oils; and mixtures thereof, with glycerol fatty acid esters and acetylated glycerol fatty acid esters are contemplated. Among the glycerol fatty acid esters, the esters comprise mono- or diglycerides, or mixtures of mono- and diglycerides, where the fatty acid moiety is a C₆ to C₂₂ fatty acid.

Also included are hydrophobic surfactants which are the reaction mixture of polyols and fatty acids, glycerides, vegetable oils, hydrogenated vegetable oils, and sterols. Polyols are polyethylene glycol, sorbitol, propylene glycol, and pentaerythritol.

Modulators of tight junction permeability include, among others, EDTA, calcium complexing agents, citric acid, salicylates, n-acyl derivatives of collagen, and enamines.

Bioadhesives include chitosan, carboxymethylcellulose, carbopol, polycarbophil, hydroxy propyl methyl cellulose, tragacanth gum and others.

Vasodilators such as nitrous oxide (NO), nitroglycerin, and arginine are included to increase blood flow in the nasal capillary bed. These include S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate.

Stabilizing delivery vehicles, carriers, support or complex-forming species include cyclodextrins, EDTA, microencapsulation systems, and liposomal formulations such as the bisphere and biosome technologies (U.S. Pat. No. 5,665,379).

Humectant or other anti-irritants are selected from compounds such as glycerol, 1,3 butanediol, tocopherol, petroleum, mineral oil, micro-crystalline waxes, polyalkenes, paraffin, cerasin, ozokerite, polyethylene, perhydrosqualene, dimethicones, cyclomethicones, alkyl siloxanes, polymethylsiloxanes, methylphenylpolysiloxanes, hydroxylated milk glyceride, castor oil, soy bean oil, maleated soy bean oil, safflower oil, cotton seed oil, corn oil, walnut oil, peanut oil, olive oil, cod liver oil, almond oil, avocado oil, palm oil, sesame oil, liquid sucrose octaesters, blends of liquid sucrose octaesters and solid polyol polyesters, lanolin oil, lanolin wax, lanolin alcohol, lanolin fatty acid, isopropyl lanolate, acetylated lanolin, acetylated lanolin alcohols, lanolin alcohol linoleate, lanolin alcohol riconoleate, beeswax, beeswax derivatives, spermaceti, myristyl myristate, stearyl stearate, carnauba and candelilla waxes, cholesterol, cholesterol fatty acid esters and homologs thereof, lecithin and derivatives, sphingolipids, ceramides, glycosphingo lipids and homologs thereof. Sodium pyroglutamate, hyaluronic acid, chitosan derivatives (carboxymethyl chitin), β-glycerophosphate, Ictamide, acetamide, ethyl, sodium and triethanolamine lactates, metal pyrrolidonecarboxylates (especially of Mg, Zn, Fe or Ca), thiamorpholinone, orotic acid, C₃-C₂₀ alpha-hydroxylated carboxylic acids, in particular α-hydroxypropionic acid, polyols, in particular inositol, glycerol, diglycerol, sorbitol, saccharide polyols, in particular alginate and guar, proteins, in particular soluble collagen and gelatin, lipoprotides chosen from mono- or polyacylated derivatives of amino acids or of polypeptides in which the acid residue RCO contains a C₁₃-C₁₉ hydrocarbon chain, in particular palmitoylcaseinic acid, palmitoylcollagenic acid, the O,N-dipalmitoyl derivative of hydroxyproline, sodium stearoylglutamate, the stearoyl tripeptide of collagen, the oleyltetra-and pentapeptide of collagen, hydroxyprolin, linoleate, uea and its derivatives, in particular xanthyl urea, cutaneous tissue extract, in particular that marketed by Laboratoires Serobiologiques de Nancy (LSN) under the name “OSMODYN®”, containing peptides, amino acids and saccharides and 17% of mannitol. A combination of glycerol, urea and palmitoylcaseinic acid is useful.

Thickeners include methylcellulose, polyvinylpyrrolidone, hydroxycellulose, chitin, sodium alginate, xanthan gum, quince seed extract, tragacanth gum, starch and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guards and the like and synthetic polymeric materials such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers polacrylates; fumed silica natural and synthetic waxes, alkyl silicone waxes such as behenyl silicone wax; aluminum silicate; lanolin derivatives such as lanesterol; higher fatty alcohols; polyethylenecopolymers; narogel; polyammonium stearate; sucrose esters; hydrophobic clays; petroleum; and hydrotalcites.

In one embodiment, the permeation-enhancement agents are selected from citric acid, sodium citrate, propylene glycol, glycerin, L-ascorbic acid, sodium metabisulfite, EDTA disodium, benzalkonium chloride and sodium hydroxide.

Preferably, the pharmaceutical composition of the invention are substantially free of native neurobiomolecules, including ganglioside, phosphatidylserine, brain-derived neurotropic factor, fibroblast growth factor, insulin, insulin-like growth factors, ciliary neurotropic factor, glia-derived nexin, cholinergic enhancing factors, phosphoethanolamine and thyroid hormone T3.

Within certain aspects of the invention, absorption-promoting agents for coordinate administration or combinatorial formulation with acetylcholinesterase inhibitor of the invention are selected from small hydrophilic molecules, including but not limited to, dimethyl sulfoxide (DMSO), dimethylformamide, ethanol, propylene glycol, 1,3 butanediol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the acetylcholinesterase inhibitor. In additional aspects, surfactants (e.g., polysorbates) are employed as adjunct compounds, processing agents, or formulation additives to enhance intranasal delivery of the acetylcholinesterase inhibitor. These penetration enhancing agents typically interact at either the polar head groups or the hydrophilic tail regions of molecules which comprise the lipid bilayer of epithelial cells lining the nasal mucosa (Barry, Pharmacology of the Skin, Vol. 1, pp. 121-137, Shroot et al., Eds., Karger, Basel, 1987; and Barry, J. Controlled Release 6:85-97, 1987, each incorporated herein by reference). Interaction at these sites may have the effect of disrupting the packing of the lipid molecules, increasing the fluidity of the bilayer, and facilitating transport of the drug across the mucosal barrier. Interaction of these penetration enhancers with the polar head groups may also cause or permit the hydrophilic regions of adjacent bilayers to take up more water and move apart, thus opening the paracellular pathway to transport of the acetylcholinesterase inhibitor. In addition to these effects, certain enhancers may have direct effects on the bulk properties of the aqueous regions of the nasal mucosa. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the acetylcholinesterase inhibitor from the vehicle into the mucosa.

Additional permeation-enhancement agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion, paracellular or intra- or trans-epithelial absorption-promoting agents that are physiologically compatible for mucosal delivery.

Other permeation-enhancement agents are selected from a variety of carriers, bases and excipients that enhance intranasal delivery, stability, activity or paracellular or trans-epithelial uptake of the acetylcholinesterase inhibitor. These include, inter alia, cyclodextrins and □-cyclodextrin derivatives (e.g., 2-hydroxypropyl-□-cyclodextrin and heptakis(2,6-di-O-methyl-□-cyclodextrin). These compounds, optionally conjugated with one or more of the active ingredients and further optionally formulated in an oleaginous base, enhance bioavailability in the pharmaceutical compositions of the invention. Yet additional permeation-enhancement agents adapted for mucosal delivery include medium-chain fatty acids, including mono- and diglycerides (e.g., sodium caprate, extracts of coconut oil, Capmul), and triglycerides (e.g., amylodextrin, Estaram 299, Miglyol 810).

The compositions of the present invention may be supplemented with any suitable permeation enhancement agent that facilitates absorption, diffusion, or penetration of acetylcholinesterase inhibitor across nasal mucosal barriers. The permeation enhancement may be any agent or system that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine, arginine etc-inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts), polyamino acids, and polycationic polymers. Also provided uptake enhancers within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Delivery of acetylcholinesterase inhibitors across the nasal mucosal epithelium can occur by a predominantly “paracellular” pathway, although some transcellular transport of the more lipophilic compounds is likely. The extent to which either paracellular or transcellular uptake dominates the overall flux and bioavailability of a drug molecule depends not only on the size of the drug molecule and its physico-chemical properties and on the excipients in the formulation, and its physical state (solid, emulsion, gel, liquid), but also on the cellar response of the nasal mucosal epithelium. Paracellular transport involves only passive diffusion, and is especially important for hydrophilic molecules smaller than 1 kilodalton (Kda), whereas transcellular transport can occur by passive, facilitated or active processes following endocytosis or membrane fusion. Generally, hydrophilic, passively transported, polar solutes, particularly small-molecular weight xenogenic chemicals (i.e., those with MW<1 KDa), diffuse through the paracellular route, while native proteins, peptides and lipophilic solutes, including hydrophobic acetylcholinesterase inhibitors, can use in part or exclusively the transcellular route of transmucosal uptake.

The nasal mucosa consists of two tissue subdomains, the olfactory membrane and the non-olfactory domain. The olfactory epithelium has a distinctive layered columnar structure containing specialized olfactory receptor cells and supporting cell types. The non-olfactory membrane is highly vascularized and the surface covered by a ciliated layered columnar epithelium. The veins of the nasal cavity drain into the superior ophthalmic vein and facial vein, which are collected in the jugular vein for return to the heart. Native neurobiomolecules with specific receptors may gain access directly across the olfactory mucosa to the cranial nerves and undergo transaxonal transport to the CNS as proposed by Frey (U.S. Pat. No. 6,180,603) but this method is limited to the upper third of the nasal turbinates and to native neurobiomolecules which are recognized for transport. Alternatively, paracellular transport into the blood and CSF is possible through tight junctions in the non-olfactory membrane and transcellular transport is possible by nonspecific endocytosis, by perturbation of lipid membranes, and by cell mediated transcytosis. We differentiate here the paracellular and transcellular transport mechanisms from the specialized transaxonal transport mechanism described by Frey and by Maitani et al. (1986. “Intranasal administration of β-interferon in rabbits,” Drug Design Delivery 1:65-70). Olfactory epithelium is concentrated in the superior nasal turbinate. Non olfactory membrane, richly vascularized, dominates in the middle and inferior nasal turbinates.

A special class of enhancers is peptides and peptidomimetics that promote nasal absorption by an unknown mechanism.

Absorption and bioavailability for passively and actively absorbed solutes can be evaluated, in terms of the sum of the paracellular and transcellular delivery components, for any selected acetylcholinesterase inhibitor within the scope of the invention. The contributions of the pathways can be distinguished according to well known methods, such as in vitro epithelial cell culture permeability assays (See, e.g., Hilgers, et al., Pharm. Res.7:902-910, 1990; Wilson et al., J. Controlled Release 11:25-40,1990; Artursson. I., Pharm. Sci. 79:476-482, 1990; Cogburn et al., Pharm. Res. 8:210216, 1991; Pade et al., Pharmaceutical Research 14:1210-1215, 1997, each incorporated herein by reference with respect to the methodologies taught therein). However, it should be cautioned that clinical studies in man are needed before extrapolating drug uptake for therapy of a clinical condition.

For passively absorbed drugs, the relative contribution of paracellular and transcellular pathways to drug transport depends upon the pKa, lipophilicity as measured crudely by the partition coefficient, molecular radius and ionic charge(s) on the drug (molecular weight has some predictive value), the pH of the luminal environment in which the drug is delivered, the buffering capacity of the formulation, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against large (i.e., greater than 5 KDa) molecular permeation would suggest that the paracellular route could be a generally less favorable route than transcellular delivery for drug transport. However, and surprisingly, the methods and compositions of the present invention provide for significantly enhanced transport of acetylcholinesterase inhibitors into and across the non-olfactory nasal mucosal epithelia via the paracellular route, with surprising increases in bioavailability relative to oral administration, and increased targeting to the central nervous system.

The pharmaceutical compositions of the invention are specially formulated for nasal delivery. With nasal breathing, nearly all particles with a size of about 10-20 um or larger are deposited on the nasal mucosa, whereas those less than 2 um can pass through the nasal cavity and be deposited in the lungs. Formulations are optimized as to their physical state and chemical composition so as to be optimally suited for intranasal delivery. Nasal formulations may be, among others: powders gels, ointments, nose drops, tampons, sponges, and sprays. Powders are dispensed with special nasal applicators. Nose sprays are dispensed by a number of devices ranging from a simple squeeze bottle to a relatively complicated piston or pump. For aqueous formulations, the viscosity and interfacial tension determines in great measure the type of device that can be used for intranasal delivery with any particular formulation. For the non-aqueous liquid formulations contemplated herein, surface tension rarely is a factor and viscosity dominates in determining expelled droplet size. The pharmaceutical compositions of the invention may be applied to one or both nasal mucosal surfaces.

In other embodiments, a viscosifier or thickener, such as a gel polymer, may be incorporated into the formulations to increase droplet size and to ensure that the pharmaceutical compostion of the invention remains in the nose. Other approaches to retaining the drug bolus in the nose include use of a higher concentration of drug, and the use of low molecular weight polyoxyethylene glycol, propylene glycols, glycerol, 1,3-butanediol, or low MW mono- and diglycerides as rapid penetrants, in combination or singularly, essentially instantaneously hydrating the nasal mucosa and thereby anchoring the formulation in the mucous layer. Suitable aqueous sprays may be delivered in a coarse particulate or droplet form, on the order of 10 to 1000 um diameter, so that the droplets go no farther than the nose. The applicator is inserted in the nasal vestibule and squeezed, a dose of the formulation usually no greater than 0.9 mL, preferably less than 0.2 mL, and most preferentially 0.1 mL, is sprayed out and deposits itself on the walls of the nose, and is immediately fixed there by the non-aqueous solvents. Although a very small amount of material can enter the oropharynx before encountering the wall of the respiratory passage, essentially no material enters the lungs. This is a condition needed for the successful use of the present invention.

Preferably, the acetylcholinesterase inhibitor is administered to the mammal in an effective dose of between about 0.1 mg and 100 mg.

Preferably, the pharmaceutical composition of the invention composition has a pH 3.0-6.0, more preferably pH 3.0-5.0 and most preferably a pH 3.0-4.0.

In some preferred embodiments, the acetylcholinesterase inhibitor is a prodrug.

In addition to the acetylcholinesterase inhibitor and permeation-enhancement agent, the pharmaceutical composition of the invention may include a pharmaceutically acceptable carrier or vehicle. As used herein, “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, pp. 1857-1859, 1990, which is incorporated herein by reference. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of intranasal administration.

The pharmaceutical compositions of the invention are generally sterile and stable for pharmaceutical use. As used herein, “stable” means a formulation that fulfills all chemical and physical specifications with respect to identity, strength, quality, and purity which have been established according to the principles of Good Manufacturing Practice, as set forth by appropriate governmental regulatory bodies.

As used herein, an “mucosally effective amount of acetylcholinesterase inhibitor” contemplates effective mucosal delivery of acetylcholinesterase inhibitor to a target site for drug activity in the subject.

In various embodiments of the invention, acetylcholinesterase inhibitor is combined with one, two, three, four or more of the permeation-enhancement agents recited in (a)-(l), above. These permeation-enhancement agents may be admixed, alone or together, with the acetylcholinesterase inhibitor, or otherwise combined therewith in a pharmaceutically acceptable formulation or delivery vehicle. Formulation of acetylcholinesterase inhibitor with one or more of the permeation-enhancement agents according to the teachings herein (optionally including any combination of two or more intranasal delivery-enhancing agents selected from (a)-(l) above) provides for increased bioavailability of the acetylcholinesterase inhibitor following delivery thereof to a nasal mucosal surface of a mammal.

In related aspects of the invention, a variety of coordinate administration methods are provided for enhanced intranasal delivery of acetylcholinesterase inhibitor. These methods comprise the step, or steps, of administering to a mammal an effective amount of at least one acetylcholinesterase inhibitor in a coordinate administration protocol with one or more intranasal delivery-enhancing agents with which the acetylcholinesterase inhibitor(s) is/are effectively combined, associated, contained, encapsulated or bound to stabilize the active agent for enhanced intranasal delivery.

To practice a coordinate administration method according to the invention, any combination of one, two or more of the intranasal delivery-enhancing agents recited in (a)-(k), above, may be admixed or otherwise combined for simultaneous intranasal administration. Alternatively, any combination of one, two or more of the mucosal delivery-enhancing agents recited in (a)-(l) can be mucosally administered, collectively or individually, in a predetermined temporal sequence separated from mucosal administration of the acetylcholinesterase inhibitor (e.g., by pre-administering one or more of the delivery-enhancing agent(s)), and via the same or different delivery route as the acetylcholinesterase inhibitor (e.g., to the same or to a different mucosal surface as the acetylcholinesterase inhibitor, or even via a non-mucosal (e.g., intramuscular, subcutaneous, or intravenous route). Coordinate administration of acetylcholinesterase inhibitor with any one, two or more of the intranasal delivery-enhancing agents according to the teachings herein provides for increased bioavailability of the acetylcholinesterase inhibitor following delivery thereof to a mucosal surface of a mammal.

In additional related aspects of the invention, various “multi-processing” or “co-processing” methods are provided for preparing formulations of acetylcholinesterase inhibitor for enhanced intranasal delivery. These methods include one or more processing or formulation steps wherein one or more acetylcholinesterase inhibitor(s) is/are serially, or simultaneously, contacted with, reacted with, or formulated with, one, two or more (including any combination of) of the permeation-enhancement agent.

To practice the multi-processing or co-processing methods according to the invention, the acetylcholinesterase inhibitor is/are exposed to, reacted with, or combinatorially formulated with any combination of one, two or more of the permeation-enhancement agent recited in (a)-(k), above, either in a series of processing or formulation steps, or in a simultaneous formulation procedure, that modifies the acetylcholinesterase inhibitor (or other formulation ingredient) in one or more structural or functional aspects, or otherwise enhances intranasal delivery of the active agent in one or more (including multiple, independent) aspect(s) that are each attributed, at least in part, to the contact, modifying action, or presence in a combinatorial formulation, of a specific intranasal delivery-enhancing agent recited in (a)-(k), above.

Many known reagents that are reported to enhance mucosal absorption also cause irritation or damage to mucosal tissues (see, e.g., Swenson and Curatolo, Adv. Drug Delivery Rev. 8:39-92, 1992, incorporated herein by reference). In this regard, the combinatorial formulation and coordinate administration methods of the present invention incorporate effective, minimally toxic delivery-enhancing agents to enhance intranasal delivery of acetylcholinesterase inhibitors useful within the invention.

While the mechanism of absorption promotion may vary with different permeation-enhancement agents of the invention, useful reagents in this context will not substantially adversely affect the mucosal tissue and will be selected according to the physicochemical characteristics of the particular acetylcholinesterase inhibitor or other active or delivery-enhancing agent. In this context, permeation-enhancement agents that increase penetration or permeability of mucosal tissues will often result in some alteration of the protective permeability barrier of the nasal mucosa. For such permeation-enhancement agents to be of value within the invention, it is generally desired that any significant changes in permeability of the nasal mucosa be reversible within a time frame appropriate to the desired duration of drug delivery. Furthermore, there should be no substantial, cumulative toxicity, nor any permanent deleterious changes induced in the barrier properties of the nasal mucosa with long-term use.

Within various aspects of the invention, improved nasal mucosal delivery formulations and methods are provided which allow delivery of acetylcholinesterase inhibitor and other therapeutic agents within the invention across mucosal barriers between administration and selected target sites. Typically, the acetylcholinesterase inhibitor is efficiently loaded at effective concentration levels in a carrier or other delivery vehicle, and is delivered and maintained in a stabilized form, e.g., at the nasal mucosa and membranes, until delivered by facilitated or passive diffusion to a remote target site for drug action (e.g., the blood stream or CNS). The acetylcholinesterase inhibitor may be provided in a delivery vehicle or otherwise modified (e.g., in the form of a prodrug), wherein release or activation of the acetylcholinesterase inhibitor is triggered by a physiological stimulus (e.g. pH change, lysosomal enzymes, etc.).

Elevated levels in CSF are taken as a good indication of therapeutic efficacy for this class of drugs. Preferably, the pharmaceutical composition of the invention following intranasal adminstration to the mammal yields a peak concentration of the acetylcholinesterase inhibitor in CSF fluid of the mammal that is greater than a nominal therapeutic concentration of the acetylcholinesterase inhibitor in the plasma of the patient. Currently accepted minimal therapeutic concentration (MTC) values in man for rivastigmine and its major active metabolite are on the order of 5 ug/L drug in CSF for 150% (half-maximal) inhibition of acetylcholinesterase activity. For donazepil in rats, MTC was reported as 0.42 nmol/gm. For tacrine in rats, MTC was reported as 3.5 nmol/gm. For TAK-147 (3-[1-(phenylmethyl)-4-piperidinyl]-1-(2,3,4,5-tetrahydro-1H-1-benzazepin-8-yl)-1-propanone) in rats, MTC was reported as 1.1 nmol/gm CSF (Kosasa T et al. 2000. “Inhibitory effect of orally administered donepezil hydrochloride (E2020), a novel treatment for Alzheimer's disease, on cholinesterase activity in rats,” Eur J Pharm 389:173-9; Bobburu J V et al. 2001. “Pharmacokinetic-pharmacodynamic modelling of rivastigmine, a cholinesterase inhibitor, in patients with Alzheimer's disease,” J Clin Pharm 41:1082-90; CutlerNR et al. 1998. “Dose-dependent CSF acetylcholinesterase inhibition by SDZ ENA 713 in Alzheimer's disease,” Acta Neurol Scand 97:244-50; Polinsky RJ. 1998. “Clinical pharmacology of rivastigmine,” Clin Ther 20:634-47).

To illustrate the methods and compositions of the invention, the following examples are included. These examples do not limit the invention. They are meant only to suggest a method of practicing the invention. Those knowledgeable in drug delivery as well as other specialties may find other methods of practicing the invention. Those methods are deemed to be within the scope of this invention.

### EXAMPLES

### Example 1

### Nasal Formulation of Donepezil

| | dry weight |
|---|---|
| Formulation | (grams) |
| Donepezil HCl | 5 |
| α-Cyclodextrin | 5 |
| Benzalkonium Chloride | 0.02 |
| Purified water | q.s. |
| pH = 4.9 | 100 mL |

### Example 2

### Nasal Formulation of Donepezil

| | dry weight |
|---|---|
| Formulation | (grams) |
| Donepezil HCl | 5 |
| Polyarginine | 0.2 |
| Benzalkonium Chloride | 0.02 |
| Purified water | q.s. |
| pH = 5.2 | 100 mL |

### Example 3

### Nasal Formulation of Donepezil

| | dry weight |
|---|---|
| Formulation | (grams) |
| Donepezil HCl | 5 |
| Benzalkonium Chloride | 0.02 |
| Purified water | q.s. |
| pH = 5.4 | 100 mL |

### Example 4

### Nasal Formulation of Donepezil

| | dry weight |
|---|---|
| Formulation | (grams) |
| Donepezil HCl | 5 |
| Chitosan | 0.5 |
| Benzalkonium Chloride | 0.02 |
| Purified water | q.s. |
| pH = 4.06 | 100 mL |

### Example 5

### Nasal Formulation of Donepezil

| | dry weight |
|---|---|
| Formulation | (grams) |
| Donepezil HCl | 5 |
| Disodium EDTA | 0.1 |
| Benzalkonium Chloride | 0.02 |
| Purified water | q.s. |
| pH = 4.6 | 100 mL |

### Example 6

### Nasal Formulation of Donepezil

| | dry weight |
|---|---|
| Formulation | (grams) |
| Donepezil HCl | 5 |
| Sodium taurocholate | 0.25 |
| Benzalkonium Chloride | 0.02 |
| Purified water | q.s. |
| pH = 5.1 (slightly hazy) | 100 mL |

### Example 7

### Nasal Formulation of Donepezil

| | dry weight |
|---|---|
| Formulation | (grams) |
| Donepezil HCl | 5 |
| Sodium taurocholate | 0.3 |
| Benzalkonium Chloride | 0.02 |
| Glycerol | 5.0 |
| Purified water | q.s. |
| pH = 5 | 100 mL |

### Example 8

### Nasal Formulation of Donepezil

| | dry weight |
|---|---|
| Formulation | (grams) |
| Donepezil HCl | 5 |
| Sodium taurocholate | 0.3 |
| Benzalkonium Chloride | 0.02 |
| Propylene Glycol | 10.0 |
| Purified water | q.s. |
| pH = 5 | 100 mL |

### Example 9

### Bioavailability in the Rat

A male rat (Rattus norvegicus Sprague-Dawley), about 180 g, was prepared surgically with an indwelling venous jugular cannula and lightly anaesthetized during the procedure. The animal was dosed intranasally in the right nostril with an intranasal formulation containing donepezil. Following dosage, the animal's head was raised to prevent the liquid from draining back out of the nasal cavity.

At 5, 10, 15, 30 and 60 min following dosage, paired blood and CSF samples were collected and placed on ice. EDTA was used as an anticoagulant and plasma was separated after centrifugation in a refrigerated centrifuge. All samples were then analyzed for donepezil by HPLC without extraction.

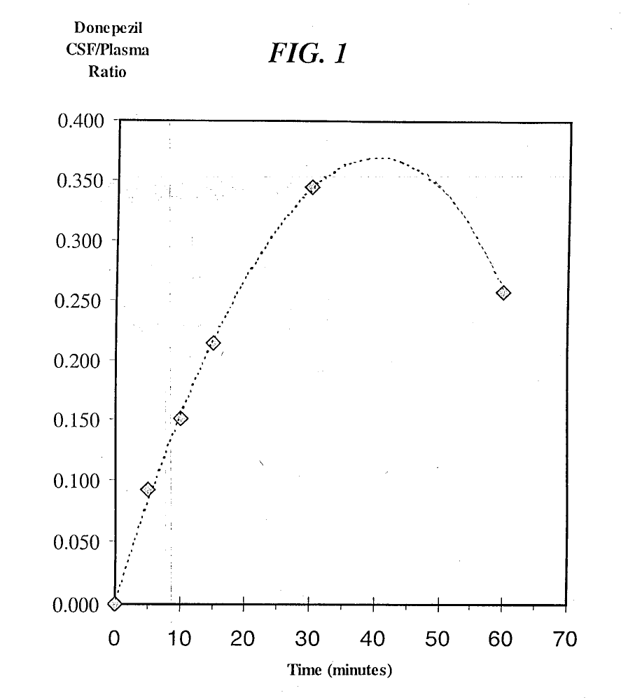

Results were plotted and are shown in FIG. 1. Donepezil is rapidly eliminated in the body by formation of inactive metabolites. However, the sharp changes in CSF to plasma ratio (peak CSF concentration 28.4 nanogram/mL at 30 min), suggests that compartment pools behave independently and that the CSF can be selectively loaded by a route independent and supplementary to plasma loading, possibly involving direct permeation to the subarachnoid plexus. We interpret this finding as a demonstration of direct CSF loading by nasal administration. We further note that the CSF concentration achieved is higher than the nominal plasma therapeutic level required for this drug.

### Example 10

### Dose tolerance in Rat

| | dry weight |
|---|---|
| Formulation | (grams) |
| Donepezil HCl | 5 |
| α-cyclodextrin | 12 |
| Purified water | q.s. |
| pH = 4.9 | 100 mL |

A formulation of donepezil 50 mg/mL in α-cyclodextrin 12% was prepared fresh for animal work. Following an approved protocol, two groups of 3 male animals each (Rattus norvegicus Lewis), 150 to 190 gm, were dosed intranasally by instilling 50 uL/kg of the test article or saline control into the right nostril. Care was taken not to damage the nasal mucosa during delivery. Following dosage, the animals head was raised to prevent the liquid from draining back out of the nasal turbinate. The dose was extrapolated from the usual human dose of 5 mg/day and on the basis of the surface area of the nasal cavity in rat versus man (7 cm² for rat versus 80 cm2 for human).

Each animal was observed continuously for 60 minutes and thereafter hourly after dosing. A staff veterinarian performed otoscopic examination of the nasal cavity prior to dosing and 4 hours after dosing.

Observations: No signs were noted of nasal irritation such as scratching of the snout, licking or biting of the snout, abnormal posturing, vocalizing, lack of motility, or any sign of pain or distress in the animals dosed with donepezil. Otoscopic examination did not reveal any difference between the saline treated animals and those treated with donepezil, although one treated animal showed slight nasal discharge from both nares immediately after dosing which resolved spontaneously in a few minutes.

It was concluded that the formulation was biocompatible and well tolerated in preclinical testing.

### Example 11

### Mucosal Deliver—Permeation Kinetics and Transmembrane Resistance

The EpiAirway system described herein was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. These are normal, human-derived tracheal/bronchial epithelial cells cultured with a proprietary medium to form a pseudo-stratified, highly differentiated tissue model which closely resembles the epithelial tissue of the respiratory tract. The apical surface is ciliated with a microvillous ultrastructure and the epithelium produces mucus (the presence of mucin has been confirmed by immunoblotting). Tight junctions have been confirmed microscopically and the tissue has a high electrical resistance characteristic of a polarized, impermeable membrane. Transepithelial resistance for the control tissue typically exceeds 550±125 ohm/cm². The inserts have a diameter of 0.875 cm, providing a surface area of 0.6 cm². The cells are plated onto the inserts at the factory approximately three weeks before shipping. One “kit” consists of 24 units. It has been shown that these differentiated primary cells are functional in paracellular transport of a plurality of drug substances and also in active transport of calcitonin, and provide valuable information predictive of in vivo behavior of nasal formulations. The test is routinely used as a screening tool for formulations and as a means of optimizing paracellular transport.

a. Prior to testing, human respiratory epithelial cells are grown to confluence in a specially designed cup designed for 6 and 24 well cell culture plates (required for testing). Note that during cell growth and differentiation prior to testing, the cells are exposed to air on the apical side and to medium on the basolateral side. A semipermeable membrane forming the base of the “cup” is used as a cell support. A proprietary medium that is cytokine-free and serum-free is used for cell growth. On arrival, the units are placed onto sterile supports in 6-well microplates. Each well receives 5 mL of proprietary culture medium. The 5 mL volume is just sufficient to provide contact to the bottoms of the units on their stands, but the apical surface of the epithelium is allowed to remain in direct contact with air. The units in their plates are maintained at 37° C. in an incubator in an atmosphere of 5% CO₂ in air for 24 hours. At the end of this time the medium is replaced with fresh medium and the units are returned to the incubator for another 24 hours.

b. In all experiments, the mucosal delivery formulation to be studied is applied to the apical surface of each “cup” containing a confluent monolayer of human respiratory epithelium in a volume of 100 μL. This volume is sufficient to cover the entire apical surface. An appropriate volume of the test formulation at the concentration applied to the apical surface (no more than 100 μL is generally needed) is set aside for subsequent determination of concentration of the drug by HPLC.

c. The units are placed in 6 well plates without stands for the experiment: each well contains 0.9 mL of pre-warmed medium which is sufficient to contact the porous membrane bottom of the unit but does not generate any significant upward hydrostatic pressure on the unit. All cells are routinely held at 37° C. in a humidified CO₂ incubator between manipulations.

d. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.

e. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.

f. At the completion of each time point, the medium is removed from the well from which each unit was transferred, and aliquotted into two tubes (one tube receives 700 μL and the other 200 μL) for determination of the concentration of permeated test material.

g. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.

h. In order to minimize errors, all tubes, plates, and wells are prelabeled before initiating an experiment. More details concerning the procedure can be found at the manufacturer's website—www.Mattek.com.

Protocol for Measurement of Transepithelial Resistance (TEER)

i. Respiratory airway epithelial cells form tight junctions in vitro as well as in vivo, restricting the flow of solutes across the tissue. These junctions confer a transepithelial resistance of several hundred ohms/cm² in excised airway tissues. We have found that the TEER of control EpiAirway units which have been sham-exposed during the sequence of steps in the permeation study is about 700-800 Ohm/cm², but, since permeation of small molecules is proportional to the inverse of the TEER, this value is sufficiently high to provide a major barrier to permeation. The porous membrane-bottomed units without cells, conversely, provide only minimal transmembrane resistance (5-20 Ohm/cm²).

ii. On arrival, the units are placed onto sterile supports in 6-well microplates. Each well receives 5 mL of proprietary culture medium. This DMEM-based medium is serum free but is supplemented with epidermal growth factor and other factors. The medium is always tested for endogenous levels of any cytokine or growth factor that is being considered for intranasal delivery, but has been free of all cytokines and factors studied to date except insulin. The 5 mL volume is just sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.

iii. The units in their plates are maintained at 37° C. in an incubator in an atmosphere of 5% CO₂ in air for 24 hours. At the end of this time the medium is replaced with fresh medium and the units are returned to the incubator for another 24 hours.

iv. Accurate determinations of TEER require that the electrodes of the ohmmeter be positioned over a significant surface area above and below the membrane, and that the distance of the electrodes from the membrane be reproducibly controlled. The method for TEER determination recommended by MatTek and employed for all experiments here employs an “EVOM”™ epithelial voltohmmeter equipped with a STX2 Electrode pair with internal Ag/AgCl reference electrodes from World Precision Instruments, Inc (Sarasota Fla.; wpiinc.com).

v. The units are read in the following sequence: all sham-treated controls, followed by all formulation-treated samples, followed by a second TEER reading of each of the sham-treated controls.

Experimental Protocol for Permeation Kinetics

i. A “kit” of 24 EpiAirway units can routinely be employed for evaluating five different formulations, each of which is applied to quadruplicate wells. Each well is employed for determination of permeation kinetics (4 time points), and transepithelial resistance. An additional set of wells is employed as controls, which are sham treated during determination of permeation kinetics, but are otherwise handled identically to the test sample-containing units for determinations of transepithelial resistance. The determinations on the controls are routinely also made on quadruplicate units.

ii. In all experiments, the mucosal delivery formulation to be studied is applied to the apical surface of each unit in a volume of 100 μL, which is sufficient to cover the entire apical surface. An appropriate volume of the test formulation at the concentration applied to the apical surface (no more than 100 μL is generally needed) is set aside for subsequent determination of concentration of the active material by HPLC, ELISA or other designated assay.

iii. The units are placed in 6 well plates without stands for the experiment: each well contains 0.9 mL of medium which is sufficient to contact the porous membrane bottom of the unit but does not generate any significant upward hydrostatic pressure on the unit.

iv. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.

v. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.

vi. At the completion of each time point, the medium is removed from the well from which each unit was transferred. These medium permeate samples are kept in the refrigerator if the assays are to be conducted within 24 hours, or the samples are subaliquotted and kept frozen at −80° C. until thawed once for assays. Repeated freeze-thaw cycles are to be avoided.

vii. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.

Results for Permeability

In vitro data was collected for permeability of donepezil as described in the protocol above. Permeability of donepezil across the tissue layer of the Epi-Airway human respiratory endothelial cell model is reported in the Table below as a mass flux in ug/min/cm². As can be seen, donepezil is very mobile in this assay.

| | | Flux |
|---|---|---|
| Exam- | | (ug/min/ |
| ple | Intranasal Delivery-Enhancing Agent | cm²) |
| 2 | 5% α-cyclodextrin + 0.02% benzalkonium Cl | 7.6 |
| 3 | 0.2% polyarginine + 0.02% benzalkonium Cl | 8.9 |
| 4 | 0.02% benzalkonium Cl | 9.4 |
| 5 | 0.5% chitosan + 0.02% benzalkonium Cl | 6.3 |
| 6 | 0.1% disodium EDTA + 0.02% benzalkonium Cl | 9.0 |
| 7 | 0.25% sodium taurocholate + 0.02% benzal- | 12.4 |
| | konium Cl | |

Note the increased flux with sodium taurocholate, a well known enhancer acting to increase paracellular transport by opening up and disrupting tight junctions and epithelial membranes.

Results for TEER

In vitro data was collected for tight junction electrical resistance in a respiratory endothelial tissue layer as described in the protocol above and is reported in the Table below.

| | | TEER |
|---|---|---|
| | | (Ohm/cm²) |
| For- | | as percent |
| mula | Intranasal Delivery-Enhancing Agent | control |
| 2 | 5% α-cyclodextrin + 0.02% benzalkonium Cl | 9 |
| 3 | 0.2% polyarginine + 0.02% benzalkonium Cl | 8 |
| 4 | 0.02% benzalkonium Cl | 6 |
| 5 | 0.5% chitosan + 0.02% benzalkonium Cl | 11 |
| 6 | 0.1% disodium EDTA + 0.02% benzalkonium Cl | 11 |
| 7 | 0.25% sodium taurocholate + 0.02% benzal- | 9 |
| | konium Cl | |

Electrical resistance of the sham treated tissue (typically about 500 to 800 Ohm/cm²) was taken as 100%. Care is taken to ensure viability of the cells exposed to each excipient. Decreased transepithelial resistance is indicative of the potency of an excipient in increasing paracellular transport.

### Example 12

### Formulation of Rivastigmine for Enhanced Intranasal Mucosal Delivery

Rivastigmine is a recently discovered acetylcholinesterase inhibitor. Prepare an exemplary formulation for enhanced mucosal delivery of rivastigmine as follows:

Formulation Composition

| Items | % mg/mL |
|---|---|
| Rivastigmine | 2.0 |
| Citric Acid Anhydrous, USP | 6.8 |
| Sodium Citrate Dihydrate, USP | 4.4 |
| 1,3 butanediol, USP | 50.0 |
| Glycerin, USP | 50.0 |
| L-Ascorbic Acid, USP | 0.12 |
| Sodium Metabisulfite, NF | 0.88 |
| Edetate Disodium, USP | 0.2 |
| Benzalkonium Chloride, NF | 0.02 |
| Sodium Hydroxide, NF or Hydrochloric Acid, NF | pH 4 |
| Purified Water, USP (q.s.) | to 100 ml |

The formulation is administered intranasally or by gastric insertion of a capsule containing the commercial formulation to 12 groups of 6 rats prepped with an indwelling jugular cannula and heparin lock. Following an approved protocol, each subject in the experimental groups is given a single dose of intranasal rivastigmine. Subsequently, each subject undergoes lumbar puncture with local anaesthesia (xylocaine s.c.), with the retrieval of 50 uL of cerebralspinal fluid (CSF). A paired blood sample from each animal is collected. By assigning groups to different timepoints, the whole PK curve can be assembled. The first CSF sample is collected 5 minutes post dosing and subsequent samples were collected at 20, 50, 75, 100 and 400 minutes. The procedure is repeated using oral rivastigmine for the control groups.

CSF samples are frozen until analysis. The data shows that rivastigmine in plasma follows a PK that is independent of the kinetics in CSF. Furthermore, when administered intranasally, the rivastigmine concentration in CSF peaks at a level higher than therapeutic levels reported for plasma following oral dosage in man. The plasma curve (AUC) for intranasal rivastigmine is shown to be remarkable by comparison to the plasma AUC for rivastigmine administered orally. We attribute this to the effects of first pass clearance on reducing AUC for drugs administered orally.

Optionally, treated animals may be tested for memory enhancement in a water maze learning model or other model of cognitive functioning.

### Example 13

### Formulation of Huperzine A (selegine) for Enhanced Intranasal Mucosal Delivery

Huperzine A is a plant derived, naturally occurring acetylcholinesterase inhibitor and is available from Sigma Chemicals (St Louis Mo.). Prepare an exemplary formulation for enhanced mucosal delivery of huperzine as follows:

Formulation Composition

| Items | % mg/mL |
|---|---|
| Huperzine A | 5.0 |
| Citric Acid Anhydrous, USP | 6.8 |
| Sodium Citrate Dihydrate, USP | 4.4 |
| Propylene Glycol, USP | 70.0 |
| Glycerin, USP | 50.0 |
| L-Ascorbic Acid, USP | 0.12 |
| Edetate Disodium, USP | 0.2 |
| Benzalkonium Chloride, NF | 0.2 |
| Sodium Hydroxide, NF or Hydrochloric Acid, NF | pH 3.5 |
| Purified Water, USP (qs) | to 100 ml |

Optionally, treated animals may be tested for memory enhancement in a water maze learning model or other model of cognitive functioning.

What is claimed is:

1. A pharmaceutical composition for treatment or prevention of a disease or condition in a mammal in need of treatment by therapeutic administration of an acetylcholinesterase inhibitor, comprising:
a. a liquid or gel solution for nasal administration of at least one acetylcholinesterase inhibitor; and
b. at least one permeation-enhancement agent for transmucosal drug uptake.

2. The pharmaceutical composition of claim 1, wherein said disease or condition is Alzheimer's disease.

3. The pharmaceutical composition of claim 1, wherein said liquid or gel solution is an aqueous solution.

4. The pharmaceutical composition of claim 1, wherein said liquid solution is a solution in a liquid polyoxyethylene glycol.

5. The pharmaceutical composition of claim 1, wherein where said liquid solution is a solution in at least one liquid selected from the group consisting of dimethylsulfoxide, n-methylpyrrolidinone, transcutol, short chain diglyceride, and short chain mononoglyceride.

6. The pharmaceutical composition of claim 1, wherein said acetylcholinesterase inhibitor is donepezil, 6-O-desmethyl donepezil, tacrine (9-amino-1,2,3,4-tetrahydroacridine hydrochloride), rivastigmine (S-n-ethyl-3-[1-dimethylamino)ethyl]-n-methyl-phyenylcarbamate hydrogen, ipidacrine, stacofylline, galanthamine, a galanthamine analog, lycoramine, a lycoramine analog, physostigmine, ambenonium, neostigmine, metrifonate, selegine, metrifonate, galanthamine, 3-[1-(phenylmethyl) piperidinyl-4-yl]-1-(2,3,4,5-tetrahydro-1H-1-benzazepine-8-yl)-1-propanone, 5,7-dihydro-3-[2-(1-(phenylmethyl)-4-piperidinyl)ethyl]-6H-pyrrolo-[4,5-f]-1,2-benzisoxazole-6-one, 4,4′-diaminodiphenylsulfone, a pyrridostigmine, a tetrahydroisoquinolinyl carbamate of a pyrroloindole, or analogs and mixtures thereof.

7. The pharmaceutical composition of claim 1, wherein said acetylcholinesterase inhibitor is donepezil or a pharmaceutically acceptable salt or a derivative thereof.

8. The pharmaceutical composition of claim 1, wherein said acetylcholinesterase inhibitor is tacrine or a pharmaceutically acceptable salt or a derivative thereof.

9. The pharmaceutical composition of claim 1, wherein said acetylcholinesterase inhibitor is rivastigmine or a pharmaceutically acceptable salt or derivative thereof.

10. The pharmaceutical composition of claim 1, wherein said acetylcholinesterase inhibitor is galantamine or a pharmaceutically acceptable salt or derivative thereof.

11. The pharmaceutical composition of claim 1, further comprising a co-therapeutic selected from the group consisting of a COX-2 inhibitor, huperzine (selegine) and 4,4′-diaminodiphenylsulfone.

12. The pharmaceutical composition of claim 1, wherein said acetylcholinesterase inhibitor is not a native neurobiomolecule.

13. The pharmaceutical composition of claim 1, wherein said pharmaceutical composition is substantially free of native neurobiomolecules selected from the group consisting of ganglioside, phosphatidylserine, brain-derived neurotropic factor, fibroblast growth factor, insulin, insulin-like growth factors, ciliary neurotropic factor, glia-derived nexin, cholinergic enhancing factors, phosphoethanolamine and thyroid hormone T3.

14. The pharmaceutical composition of claim 1, wherein said pharmaceutical composition is substantially free of G-1 ganglioside.

15. The pharmaceutical composition of claim 1, wherein said pharmaceutical composition is substantially free of native neurobiomolecules that stimulate nerve cell growth.

16. The pharmaceutical composition of claim 1, wherein said permeation-enhancement agent is selected from:
(a) an aggregation inhibitory agent;
(b) a charge modifying agent;
(c) a pH control or buffering agent;
(d) a redox control or buffering agent
(e) a degradative enzyme inhibitory agent;
(f) a mucolytic or mucus clearing agent;
(g) a ciliostatic agent;
(h) an absorption enhancement agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or β-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(i) a modulatory agent of epithel ial junction physiology;
(j) a vasodilator agent;
(k) a stabilizing delivery vehicle, carrier, support or complex-forming species with which the acetylcholinesterase inhibitor is effectively combined, associated, contained, encapsulated or bound resulting in stabilization of the acetylcholinesterase inhibitor for enhanced mucosal delivery, wherein the formulation of said acetylcholinesterase inhibitor with said one or more delivery-enhancing agents provides for increased bioavailability of the acetylcholinesterase inhibitor in a central nervous system tissue or fluid of said subject; and
(l) a humectant or membrane stabilizing agent.

17. The pharmaceutical composition of claim 1, comprising a plurality of said permeation-enhancement agents.

18. The pharmaceutical composition of claim 1, comprising a plurality of said absorption enhancing agents.

19. The pharmaceutical composition of claim 1, wherein said absorption enhancing agent is glycyrrhitic acid or a derivative thereof.

20. The pharmaceutical composition of claim 1, further comprising a chitosan or chitosan derivative.

21. The pharmaceutical formulation of claim 20, wherein said chitosan or chitosan derivative is poly-GuD.

22. The pharmaceutical composition of claim 1, wherein said pharmaceutical composition has a pH 3.0-6.0.

23. The pharmaceutical composition of claim 1, wherein said pharmaceutical composition has a pH 3.0-5.0.

24. The pharmaceutical composition of claim 1, wherein said pharmaceutical composition has a pH 3.0-4.0.

25. The pharmaceutical composition of claim 1, wherein said acetylcholinesterase inhibitor is administered to said mammal in an effective dose of between about 0.1 mg and about 100 mg.

26. The pharmaceutical composition of claim 1, wherein said permeation-enhancement agent is selected from the group consisting of citric acid, sodium citrate, propylene glycol, glycerin, L-ascorbic acid, sodium metabisulfite, edetate disodium, benzalkonium chloride, sodium hydroxide and mixtures thereof.

27. The pharmaceutical composition of claim 1, wherein said acetylcholinesterase inhibitor is a prodrug.

28. The pharmaceutical composition of claim 1, further comprising a membrane stabilizing agent to reduce nasal irritation.

29. The pharmaceutical composition of claim 1, wherein said membrane stabilizing agent to reduce nasal irritation is vitamin E or a derivative of vitamin E.

30. The pharmaceutical composition of claim 1, wherein said pharmaceutical composition following intranasal adminstration to said mammal yields a peak concentration of said acetylcholinesterase inhibitor in a central nervous system tissue or fluid of said mammal that is at least equal to a therapeutic plasma concentration of said acetylcholinesterase inhibitor in a blood plasma of said mammal.

31. The pharmaceutical composition of claim 30, wherein said acetylcholinesterase inhibitor is donepezil or a pharmaceutically acceptable salt or derivative thereof.

32. The pharmaceutical composition of claim 30, wherein said acetylcholinesterase inhibitor is tacrine or a pharmaceutically acceptable salt or a derivative thereof.

33. The pharmaceutical composition of claim 30, wherein said acetylcholinesterase inhibitor is rivastigmine or a pharmaceutically acceptable salt or derivative thereof.

34. The pharmaceutical composition of claim 1, wherein said nasal administration involves delivery of said composition to one or both nasal mucosal surfaces of said mammal.

35. A method for treating or preventing a disease or condition in a mammal in need of treatment by therapeutic administration of an acetylcholinesterase inhibitor, comprising the step of administering intranasally to said mammal a pharmaceutical composition of claim 1.

36. The method of claim 35, wherein said pharmaceutical composition is administered as a single solution in a multidose nasal dispenser.

37. The method of claim 35, wherein said disease or condition amenable to treatment by therapeutic administration of said acetylcholinesterase inhibitor is Alzheimer's disease.

38. The method of claim 35, wherein said disease or condition amenable to treatment by therapeutic administration of said acetylcholinesterase inhibitor is Parkinson's-like dementia.

39. The method of claim 35, wherein said disease or condition amenable to treatment by therapeutic administration of said acetyicholinesterase inhibitor is Huntington's-type dementia.

40. The method of claim 35, wherein said disease or condition amenable to treatment by therapeutic administration of said acetylcholinesterase inhibitor is Pick's-type dementia.

41. The method of claim 35, wherein said disease or condition amenable to treatment by therapeutic administration of said acetylcholinesterase inhibitor is AIDS related dementia or delirium.

42. The method of claim 35, wherein said disease or condition amenable to treatment by therapeutic administration of said acetylcholinesterase inhibitor is dementia secondary to vascular disorder.

43. The method of claim 35, wherein said disease or condition amenable to treatment by therapeutic administration of said acetylcholinesterase inhibitor is moderate cognitive impairment.

44. The method of claim 35, wherein said disease or condition amenable to treatment by therapeutic administration of said acetylcholinesterase inhibitor is Crutzfeld-Jacobsen type dementia.

45. The method of claim 35, wherein said disease or condition amenable to treatment by therapeutic administration of said acetylcholinesterase inhibitor is a learning disorder.

46. The method of claim 35, wherein said disease or condition amenable to treatment by therapeutic administration of said acetylcholinesterase inhibitor is nicotine withdrawal syndrome.

47. The method of claim 35, wherein said administration involves delivery of said pharmaceutical composition to a nasal mucosal surface of said mammal.

48. The method of claim 35, wherein said acetylcholinesterase inhibitor is tacrine or a pharmaceutically acceptable salt or derivative thereof.

49. The method of claim 35, wherein said acetylcholinesterase inhibitor is rivastigmine or a pharmaceutically acceptable salt or derivative thereof.

50. The method of claim 35, wherein said acetylcholinesterase inhibitor is administered to said mammal in an effective dose of between about 0.1 mg and 100 mg.

51. The method of claim 35, wherein said permeation-enhancement agent for transmucosal drug uptake is selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or (x-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x);
(h) a modulatory agent of epithelial junction physiology;
(i) a vasodilator agent;
(j) a selective transport-enhancing agent; and
(k) a stabilizing delivery vehicle, carrier, support or complex-forming species with which the acetylcholinesterase inhibitor is effectively combined, associated, contained, encapsulated or bound resulting in stabilization of the acetylcholinesterase inhibitor for enhanced mucosal delivery, wherein the formulation of said acetylcholinesterase inhibitor with said one or more delivery-enhancing agents provides for increased bioavailability of the acetylcholinesterase inhibitor in a central nervous system tissue or fluid of said subject, and,
(m) a humectant or membrane stabilizing agent.

52. The method of claim 35, wherein said permeation-enhancement agent is selected from the group consisting of citric acid, sodium citrate, propylene glycol, glycerin, L-ascorbic acid, sodium metabisulfite, edetate disodium, benzalkonium chloride, sodium hydroxide and mixtures thereof.

53. The method of claim 35, wherein said pharmaceutical composition following intranasal adminstration to said mammal yields a peak concentration of said acetylcholinesterase inhibitor in a central nervous system tissue or fluid of said mammal that is at least 10% of the peak concentration of said acetylcholinesterase inhibitor in a blood plasma of said mammal.

54. The method of claim 35, wherein said pharmaceutical composition following intranasal adminstration to said mammal yields a peak concentration of said acetylcholinesterase inhibitor in a central nervous system tissue or fluid of said mammal that is at least 15% of the peak concentration of said acetylcholinesterase inhibitor in a blood plasma of said mammal.

55. The method of claim 35, wherein said pharmaceutical composition following intranasal adminstration to said mammal yields a peak concentration of said acetylcholinesterase inhibitor in a central nervous system tissue or fluid of said mammal that is at least 20% of the peak concentration of said acetylcholinesterase inhibitor in a blood plasma of said mammal.

56. The method of claim 35, wherein said pharmaceutical composition following intranasal adminstration to said mammal yields a peak concentration of said acetylcholinesterase inhibitor in a central nervous system tissue or fluid of said mammal that is at least 25% of the peak concentration of said acetylcholinesterase inhibitor in a blood plasma of said mammal.

57. The method of claim 35, wherein said pharmaceutical composition following intranasal adminstration to a mammal yields a peak concentration of said acetylcholinesterase inhibitor in a central nervous system tissue or fluid of said mammal that is at least 30% of the peak concentration of said acetylcholinesterase inhibitor in a blood plasma of said mammal.

58. The method of claim 35, wherein said pharmaceutical composition following intranasal adminstration to said mammal yields a peak concentration of said acetylcholinesterase inhibitor in a central nervous system tissue or fluid of said mammal that is at least 40% of the peak concentration of said acetylcholinesterase inhibitor in a blood plasma of said mammal.

59. The method of claim 35, wherein said formulation following mucosal adminstration to said mammal yields a peak concentration of said acetylcholinesterase inhibitor in a central nervous system tissue or fluid of said subject that is greater than a therapeutic concentration of said acetylcholinesterase inhibitor in the plasma of said subject.

60. An article of manufacture, comprising:
a. a means for administering a nasal dose; and
b. the composition of claim 1.

61. The article of manufacture of claim 60, wherein said means for administering a nasal dose is a nasal dispenser, tampon, sponge, insufflator, nebulizer or pump.

62. An article of manufacture comprising the pharmaceutical composition of claim 1 in a package suitable for sale and distribution.
